(19) United States
(12) Patent Application Publication
(10) Pub. No.: US 2026/0151181 A1
(43) Pub. Date: Jun. 4, 2026

(54) DEVICES AND METHODS USING MAGNETIC FORCES IN MANIPULATING COOPERATIVE ABLATION ELEMENTS

(71) Applicant: The Board of Trustees of the Leland Stanford Junior University, Standord, CA (US)

(72) Inventors: Paul J. Wang, Saratoga, CA (US); Meghedi Babakhanian, Sunland, CA (US); Anson Lee, Redwood City, CA (US); Terrence Pong, Foster City, CA (US); Eajer Toh, Redwood City, CA (US)

(21) Appl. No.: 18/033,763

(22) PCT Filed: Oct. 26, 2021
(86) PCT No.: PCT/US2021/056679

§ 371 Date: Apr. 25, 2023

Related U.S. Application Data
(60) Provisional application No. 63/105,777, filed on Oct. 26, 2020.

(51) Int. Cl. A61B 18/14; A61B 18/00; A61B 18/16

(52) U.S. Cl. CPC A61B 18/1492; A61B 18/16; A61B 2018/00184; A61B 2018/00357; A61B 2018/00363; A61B 2018/00577; A61B 2018/00642; A61B 2018/00702

(57) ABSTRACT

In certain examples, an epi instrument an ablation-delivery element and at least one epi magnet secured relative to the ablation-delivery element, and an endo instrument has at least one endo magnet and is configured with the endo magnet to facilitate positioning before ablation. The instruments are moved towards a first target ablation site, and then to a second ablation site, while the at least one ablation-delivery element and the endo instrument are drawn towards one another via magnetic forces and on either sides of tissue structure. The ablation-delivery element and the endo instrument are cooperatively configured to move together via the magnetic forces for the ablation. In more specific examples, the epi-endo system may use the epi-endo magnetic array to maintain a consistent contact force independent of the tissue thickness and/or may use a sensor feedback and controlled energy arrangement to provide feedback concerning the thickness(es) of the tissue area(s) targeted for the ablation.

### FEDERALLY-SPONSORED RESEARCH AND DEVELOPMENT

This invention was made with Government support under contract TR003142 awarded by the National Institutes of Health. The Government has certain rights in the invention.

### BACKGROUND

Aspects of the present disclosure are related generally to ablation devices, systems and methods as may be applied to treat cardiac arrhythmias and other target sites where application of one or more types of ablation may be applied for medical treatment.

In exemplary contexts, aspects of the present disclosure are directed to ablation of tissues such as in the heart, liver, etc. For example, atrial fibrillation (AF) is a common irregular heartbeat, affecting 3-5 million Americans. AF is associated with significant burden in terms of morbidity and mortality from stroke, heart failure, and impaired quality of life translating to significant effects on healthcare costs and resource use. Despite progress in pharmacologic therapy and multiple catheter design advancements such as tissue contact force and cryoablation, the AF ablation single procedure success rate remains below 50% at one year and less than 20% at five years in patients with persistent AF. On the other hand, the open heart surgery Cox-Maze procedure, is capable of achieving 80-90% success at one year and 70% success at five years by creating lasting surgical incisions. However, almost no patients are willing to undergo such an invasive procedure. Thus, there remains a significant clinical need for patients with persistent AF.

Using heart tissue as one such application type for ease of discussion, it has been appreciated that cardiac-directed catheter ablation is a technique to treat cardiac arrhythmias by creating cell damage. Most forms of catheter ablation for arrhythmias are performed by delivering energy from the endocardial surface of the heart. Only a small proportion of ablation procedures are performed from the epicardial surface of the heart. Surgical ablation of arrhythmias may be performed endocardially and epicardially, but only epicardial ablation may be performed without cardiopulmonary bypass.

While epicardial ablation may be successful in some regions of the heart, in many cases, transmural lesions may be difficult to achieve using an epicardial approach alone. Current approaches to epicardial and endocardial ablation have limitations. One limitation relates to the difficulty of positioning the ablation electrode and achieving adequate contact with the tissue surface that may limit the precision and ease of mapping such as for obtaining electrical signals from the heart that may identify the site of ablation.

Another limitation relates to the tissue thickness at the target site of the ablation. For example, the tachycardia focus may require transmural lesions which may be difficult to achieve from either the endocardial or epicardial surface of the heart alone.

Yet another limitation relates to being able to track the position of an ablation instrument. In connection with certain arrhythmia treatments, for example, tracking the movement within a patient of one or multiple instrument(s) as may be required for an ablation procedure may be difficult. While one such instrument is being moved for accurate positioning (e.g., as mapped) and tracking of the same instrument or another instrument can be at least momentarily interrupted, this may delay or complicate the ability to treat a certain tissue area in real time such as when certain cardia nerve sites fire unexpectedly for a short period of time.

Still another limitation relates to the fact that obtaining two separate maps of the endocardial and epicardium is time consuming and does not permit one to align the epi and endo instruments as may be needed in a timely and accurate manner such as for endocardial lesions to create a transmural lesion.

### SUMMARY OF VARIOUS ASPECTS AND EXAMPLES

Various aspects and examples according to the present disclosure (including the Appendix filed as part of the underlying provisional application) are directed to issues such as those addressed above and/or others which may become apparent from the following disclosure involving a system and/or approach in which target tissue to be ablated is addressed from two opposing sides (e.g., as applied to heart tissue, both epicardial and endocardial sides) and to induce full thickness ablations. This dual-sided approach is to avoid recurrences, for example, of AFIB when applied to the heart.

In certain specific examples, a pair of epi and endo instruments are part of a system that control an epi-endo magnetic array to maintain a consistent contact force independent of the tissue thickness associated with the ablation. This aspects may, optionally, use a sensor feedback and controlled energy arrangement to provide feedback concerning the tissue thickness which is targeted for the ablation.

In specific examples, apparatuses and/or methods for using such apparatuses involve an epi instrument, an ablation-delivery element and at least one epi magnet secured relative to the ablation-delivery element, and the apparatus further includes an endo instrument having at least one endo magnet (e.g., as part of a rounded-shape mechanism configured with the endo magnet to facilitate endo-instrument movement and positioning).

The instruments are capable of being moved towards a first target site, and then to a second site, while the at least one ablation-delivery element and the endo instrument are drawn towards one another via magnetic forces and on either side of tissue structure. The ablation-delivery element and the endo instrument are cooperatively configured to move together via the magnetic forces for the ablation.

According to one specific method-related example, the following steps are carried out: (a) introduction of a guiding mechanism (e.g., a cable, rail, lumen or catheter) attached to an ablation unit or assembly as may be used to set up for an alignment system for respectively opposing magnetic polarities; (b) introduction of at least one part of the system into a catheter (e.g., endo-ablation catheter) so as to facilitate magnetic pairing of the epi-and endo-elements; and (c) movement of the epi-ablation element along or via the guiding mechanism until reaching a target site where an ablation application may be carried out. Optionally, the epi and endo instruments, or at least the ablation electrode(s) and the endo instrument, are capable of being cooperatively moved towards and from the first target site for ablation at a different site.

In certain other more-specific examples which may also build on the above-discussed aspects, such apparatuses may involve a control circuit, including logic circuitry, to control the magnetic forces (e.g., as a function of thickness of the tissue structure), and wherein the mechanism refers to or includes: a spherical-like basket containing the at least one endo magnet or at least one additional endo magnet having a spherical or cylindrical shape.

In related specific examples related to the above apparatuses, further aspects may involve use of a spherical-like basket, the at least one epi magnet and the at least one ablation-delivery element. The (at least one) ablation-delivery element may be fixedly secured by an optional guiding mechanism (e.g., a rail) while used in a tissue-presentation mode which precedes a mode in which the at least one ablation-delivery element is to apply energy for ablating cells, and/or may be moved along a pathway or guiding mechanism to facilitate the movement of the at least one ablation-delivery element towards a second target ablation while magnetic forces remain engaged between the at least one epi magnet and the at least one endo magnet. In one such specific example, an endo catheter inserted via the femoral vein and a sheath may be used to introduce the endo instrument via the endo catheter.

According to other more-specific examples related to certain of the above apparatuses wherein the mechanism has at least one additional (spherically- or cylindrically-shaped) endo magnet, each of the at least one endo magnet and the additional endo magnet may form a series of magnets configured as series of magnets in an array which is configured and/or used to augment a magnetic field on one side of the array (e.g., such an array may be in a Halback arrangement or Poly arrangement), and wherein the ablation-delivery element may include an ablation-energy-delivery component, or electrode, secured to respective surfaces of a plurality of immediately adjacent ones of the set of magnets arranged in a sequence.

In yet another more specific aspect related to the above exemplary apparatuses, aspects of the present disclosure are directed to a method including at least the following steps: cooperatively positioning a set of ablation instruments including an epi instrument having a guiding mechanism (e.g., a cable, rail, lumen or catheter), at least one ablation-delivery element and at least one epi magnet secured relative to the at least one ablation-delivery element, and an endo instrument having at least one endo magnet and a mechanism including at least one structure with a rounded shape, and being configured with the at least one endo magnet to facilitate positioning of the endo instrument; applying a force to cause the guiding mechanism and the at least one ablation-delivery element to move towards a first target site, where nearby cells are to be ablated while the at least one ablation-delivery element and the endo instrument are drawn towards one another via magnetic forces and on either sides of tissue structure; ablating, via the at least one ablation-delivery element and the endo instrument, cells near the first target site while the magnetic forces draw the at least one ablation-delivery element and the endo instrument towards one another while being located on either sides of tissue structure; causing cooperative movement of the at least one ablation-delivery element and the endo instrument towards a second target site while magnetic forces are engaged between the at least one epi magnet and the at least one endo magnet; and using the at least one ablation-delivery element to ablate cells in tissue structure near the second target site while the epi instrument and the endo instrument are on opposing sides of the tissue structure near the second target site.

According to yet further exemplary embodiments, in place of the magnets or to augment the opposing magnet forces provided by the magnets on either side of the tissue, the system may include a vacuum and in some instances, the system may also include or include as another alternative magnetic-like mechanism an inflatable balloon (or a balloon on each side of the tissue) to press the ablation elements against the tissue in a restricted region.

The above discussion is not intended to describe each aspect, embodiment or every implementation of the present disclosure. The figures and detailed description that follow also exemplify various embodiments.

### BRIEF DESCRIPTION OF FIGURES

Various example embodiments, including experimental examples, may be more completely understood in consideration of the following detailed description in connection with the accompanying drawings, each in accordance with the present disclosure, in which:

FIG. 1 is a flow diagram depicting one example of an ablation-directed apparatus, according to certain exemplary aspects of the present disclosure, may be used;

FIGS. 2A and 2B respectively show an endo instrument and an epi instrument of one type of exemplary ablation-directed apparatus, according to certain exemplary aspects of the present disclosure;

FIGS. 3A, 3B and 3C are related diagrams respectively showing steps of another example for use of an ablation-directed apparatus, consistent with the type of apparatus shown in FIGS. 2A and 2B, as may be applied in a cardia procedure and also according to the present disclosure;

FIGS. 4A, 4B and 4C are perspective views of an exemplary apparatus with parts of epi instruments which may be used in connection with another type of exemplary ablation-directed apparatus, according to certain exemplary aspects of the present disclosure;

FIG. 5 is a heart-related diagram illustrating application, from an endocardium perspective, of using the second type of apparatus showing pathways for treatment in connection with left atrium (LA) posterior box lesion, according to certain exemplary aspects of the present disclosure;

FIGS. 6A, 6B, 6C, 6D and 6E are related diagrams (in X-ray form) depicting catheter application of the second type of apparatus with each of these diagrams showing sequential progress of the apparatus' epi (or endo) instrument being moved through a patient to a complementarily-constructed (or epi) instrument for an epi-endo-paired positioning on either side of targeted tissue before delivery of ablation energy, according to certain exemplary aspects of the present disclosure; and

FIGS. 7A and 7B are respective graphs showing different contact force studies validating performance criteria in an experiment using another type of exemplary ablation-directed apparatus, with FIG. 7A showing various forces versus distance associated with different magnetic-type structures, and FIG. 7B showing a five-element ring-array design versus a seven-element ring-array design, according to certain exemplary aspects of the present disclosure.

While various embodiments discussed herein are amenable to modifications and alternative forms, aspects thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of different types of apparatuses, systems and methods involving devices characterized in part by cooperatively-designed set of instruments (e.g., an epi instrument and an endo-instrument) for surgical application of ablation energy via one or more ablation electrodes. As such surgical application may involve various treatments of cardiac arrhythmia, this form of treatment is exemplified in connection with many of the figures so as to facilitate better understanding of the various examples disclosed in the present disclosure. Accordingly, while the present disclosure is not necessarily limited to such aspects and treatment, an understanding of specific examples in the following description may be understood from discussion in such specific contexts.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same connotation and/or reference numerals may be used in different diagrams to refer to the same elements or additional instances of the same element. Also, although aspects and features may in some cases be described in individual figures, it will be appreciated that features from one figure or embodiment can be combined with features of another figure or embodiment even though the combination is not explicitly shown or explicitly described as a combination.

In certain example embodiments, aspects of the present disclosure involve an ablation procedure intended for a selectable thickness, e.g., up to a full thickness ablation across the targeted tissue, for example, the cardiac tissue. In such a cardiac context, ideally, the use of two cooperative portions of the apparatus provides for perfect alignment of the epi and endo ablator catheters across the tissue where bipolar energy is delivered only across the tissue at the target. This approach may be used to create a close loop feedback system. The feedback may be used to sense and/or indicate where one aspect of the device is located within the patient (e.g., approaching or in proximity to the target tissue) so as to provide direction or an indication of when to stop the insertion procedure as may be applied to one or the other, or each, of the portions of the devices being inserted.

In a more-specific example, one of the cooperative portions of the apparatus employs a series of magnets on, in or along a structure, such that the structure will self-align and enable automatic self-alignment of the other cooperative portion, such as catheters used for the ablation. A series of ablation elements may be in communication with endo elements to adjust ablation intensity (time) depending on the tissue thickness. This may be used to create a close loop feedback system. The feedback may be used to sense and/or indicate where one aspect of the device is located within the patient (e.g., approaching or in proximity to the target tissue) so as to provide direction or an indication of when to stop the insertion procedure as may be applied to one or the other, or each, of the portions of the devices being inserted.

In one particular example, the present disclosure is directed to a method involving an array ablation unit, for cardiac-treatment, configured to act on both epicardial and endocardial sides. In one array-based cryoablation design, one or more arrays are designed to completely form a loop, or in a shorter length that will cover a portion of the loop, and will move along a path to complete the arc or loop. Ablation sources and material may include: RF Electroporation Ultrasound Cryoablation, and other sources of ablation may include a laser with software for programming a CPU to provide control. In such specific embodiment (a single ablation element design), a feedback system is to adjust magnetic strength as tissue thickness varies across the ablation path. There may be provided as part of the feedback system impedance associated with the adjacent tissue(s) and contact force between the tissue and the ablation element/elements, and magnetic field strength between the magnet pairs across the tissue. Also, the array-based ablation design feedback system may be configured to monitor impedance and temperature per ablation element, thereby creating a closed-loop ablation system to address ablation time variation needed per tissue thickness across the ablation path.

In one specific design example, an endocardial assembly includes an ablation element, a magnetic element and an expandable element that when expanded surrounds the ablation and magnetic elements. The epicardial assembly includes an ablation element and a magnetic element that magnetically attracts the magnetic element of the endocardial assembly and allows for alignment of the epicardial assembly with the endocardial assembly on opposite sides of heart tissue. Movement of the epicardial assembly causes passive, smooth movement of the endocardial assembly along an inner surface of the heart.

In another specific design example, both endocardial and epicardial array assemblies include the following components: arrangement of magnets in an orientation and arrangement that pairs epi and endo arrays to each other in one correct direction. The magnets can be arranged in multiple settings to create the perfect pairing between the epi and endo units across the tissue; ablation element or elements in contact with the tissue can be a coating on the solid magnets, it can be a solid material, an accordion shape mesh, stent like or a mesh like material conductive to RF and other energies; and optionally, the assembly can have a guiding mechanism (e.g., tube, rail, etc.) to be situated around the pulmonary veins and for directing the epicardial ablation array in the ablation path while the endocardial array follows and pairs.

Endocardial distal end array tubing may be made using very soft material (e.g., semi-rigid plastic) to ensure flexibility and hard to reach targets. The irrigation system and thermocouples may be incorporated into each ablation element, and in the array design, impedance measurement across the tissue may be used to develop a closed loop feedback system and ensure consistent ablation with respect to varying tissue thickness along the ablation path. The ablation element can be one unit or a series of electrodes in the line of ablation. Each individual epicardial electrode can communicate with a single endo unified ground or their respective ground endo electrode to ablate across the tissue based on respective tissue thickness. Other components of the catheter system may be a sensor such as an infrared (IR) sensor or impedance measurement to verify correct alignment of the arrays prior to ablation. By having the distal end of the catheter designed to be hyper flexible (e.g., using a sufficiently polymer material), the magnetic array could be realized so as to be sufficiently flexible at least near its distal end portion via any of different designs such as: a flexible polymer, a ball bearing joint system, very thin coiled tube/lumen extension housing the cables and/or irrigation.

Although the embodiments shown include magnets, other mechanisms for moving, positioning, attracting and aligning ablation element in two catheters across the heart tissue may also be used in conjunction with the magnetic forces on its own (e.g., without a rail or other guiding mechanism). For example, other mechanisms with alignment abilities include, but are not limited to, ultrasound, sensors (e.g. IR), and GPS sensors. Such embodiments may include, for example, an endocardial catheter with at least a distal end portion or tip that is sufficiently flexible (e.g., hyperflexible) such that the portion of the catheter is able to bend at approximately ninety degree angles (plus or minus 5-10%). In such detailed examples, rather than implementing with the endo-catheter with a stiff steering ability, via stiffness at catheter's distal end, by using a flexible or hyperflexible) endocardial catheter, as above, access to all target areas is readily facilitated. In this manner, the endo-catheter distal tip does not limit ablation only to those areas at which a still catheter may provide access (e.g., resulting in incomplete ablations or gaps in ablation paths which increases the risk of AF reoccurrence). Rather the flexible or hyperflexible distal tip of the endocardial catheter allows for seamless pairing with the epi-array and permits for magnetic pull (e.g., the epi-unit to easily follow) across tissue along the targeted path for the ablation event(s). This smooth movement from the endocardial side allows the flexible catheter, according to such specific embodiments of the present disclosure, to reach hard to access targets and ensure a continuous and gap-free ablation path.

Other magnetic-like forces, which may also be used such as alternatives to illustrated examples of such magnets, may be provided to augment and/or in place of the above-discussed magnetic attraction methodology. These forces may be realized by vacuum and/or other mechanisms including inflatable balloons to press the ablation elements against the tissue in a restricted region.

According to more specific embodiments of the present disclosure, such flexibility or hyper flexibility may only applies to the distal end of the catheter following the magnetic array (as opposed to other portions such as that extending over/past the magnetic array. The (hyper) flexibility may also be achieved via different designs such as a flexible polymer, a ball bearing joint system, and/or a very thin coiled tube/lumen extension housing the cables.

Also in accordance with a number of embodiments of the present disclosure (e.g., associated with a type of design which may be referred to as Design type 1), magnetic force between the magnetic elements of the epicardial and endocardial assemblies may be adjustable. In other related embodiments, the endocardial assembly may include an expandable element that when expanded allows the endocardial assembly to move smoothly along endocardial tissue surface. In still further embodiments, the endocardial assembly is configured and arranged such that a portion thereof is capable of rotating about the magnetic portion as it moves along the inner surface of the heart.

In connection with another type of design (e.g., Design type 2) and also in accordance with a number of embodiments of the present disclosure, magnetic force between the magnetic elements of the epicardial and endocardial array assemblies may be adjustable or stay constant. If the magnetic strength stays constant, the ablation time will be altered according to tissue thickness to ensure consistent ablation. In still further embodiments, the endocardial assembly is configured and arranged such that a portion thereof is capable of expanding about the array of magnetic array portion after it is advanced inside the left atria. The endocardial unit aligns and follows the epicardial array along the inner surface of the heart. In another embodiment, a sensor such as an IR sensor can be used to validate ablation array correct alignment across the tissue prior to ablation.

Consistent with the above aspects, such a manufactured device or method of such manufacture may involve aspects presented and claimed in U.S. Provisional, Application Ser. No. 63/105,777 filed on Oct. 26, 2020(STFD.423P1 ), to which priority is claimed. To the extent permitted, such subject matter is incorporated by reference in its entirety generally and to the extent that further aspects and examples (such as experimental and/more-detailed embodiments) may be useful to supplement and/or clarify.

Consistent with the present disclosure, one particular aspect of the present disclosure is directed to more specific types of methods involving use of the types of epi-and endo-instruments as described above. In one such method, the following steps are carried out: (a) introduction of a guiding mechanism (e.g., a rail, cable) attached to an ablation unit or assembly (e.g., a may be used to set up for respectively opposing magnetic polarities); (b) introduction of at least one part into a catheter (e.g., a part of the endo-unit endo-ablation catheter) so as to facilitate magnetic pairing of the epi-and endo-elements; and (c) movement of the epi-ablation element along the ablation path or guiding mechanism (e.g., rail) until reaching a target site where an ablation application may be carried out.

The following discussion is directed to another specific type of method according to the present disclosure, and also involving use of the types of epi-and endo-instruments designed to be cooperatively positioned, as described above. As such, the approach involves cooperatively positioning a set of ablation instruments including an epi instrument having at least one ablation-delivery element and at least one epi magnet secured relative to the at least one ablation-delivery element, and an endo instrument having at least one endo magnet and an elongated portion with a rounded or curved shape (e.g., endo catheter, presentation end of magnetic array), and being configured with the at least one endo magnet to facilitate positioning of the endo instrument. Next, application of a force causes the endo and epi aspects (e.g., the at least one endo magnet and the at least one ablation-delivery element) to move towards a first target site, where nearby cells are to be ablated while the at least one ablation-delivery element and the endo instrument are drawn towards one another via magnetic forces and on either sides of tissue structure.

Once positioned, or in some instances during slight movement, the approach continues by ablating, via the at least one ablation-delivery element and the endo instrument, cells near the first target site while the magnetic forces draw the at least one ablation-delivery element and the endo instrument towards one another while being located on either sides of tissue structure. Further, cooperative movement of the at least one ablation-delivery element and the endo instrument is effected to directed the instrument(s) towards a second target site while magnetic forces are engaged between the at least one epi magnet and the at least one endo magnet. In tissue structure near the second target site, the approach then involves using the at least one ablation-delivery element to ablate cells while the epi instrument and the endo instrument are on opposing sides of the tissue structure near the second target site. As noted above, by cooperatively moving the relevant parts of the instruments to such opposing tissue sides, in the case of heart tissue referring to both epicardial and endocardial sides, concerns associated with tissue thickness are more readily overcome and controlled. As examples,. with use of a controller delivery of appropriate amounts of RF ablation energy on a focused portion of target tissue may be in ranges characterized according to known standards.

According to other aspects, the above type of approach may further involve the endo instrument (initially contracted during presentation such as during movement in an endo catheter and while positioning before ablation) having an expandable, rotatable basket in which the at least one endo magnet resides, wherein the expandable, rotatable basket rotates and facilitates the at least one endo magnet to be pulled along by the at least one epi magnet in response to the magnetic forces. Also, while the at least one ablation-delivery element applies energy to ablate the cells, the expandable, rotatable basket may be used as a ground reference such as for a radio-frequency (RF) ablation requiring a reference return path for electrical current flowing through the cells via the ablation electrode(s).

In various applications, such approaches may involve or include introducing the epi instrument thoracoscopically while the at least one ablation-delivery element and the at least one epi magnet are secured to as part of the endo instrument. In a further aspect relating to one or more of the above approaches where applicable or advantageous, the apparatus (including the epi and endo instruments) may include and/or involve use of a vacuum to draw the epi instrument and/or endo instruments (or rail in which the epi instrument may be positioned in certain cases) against the tissue structure. In this manner, one of the instruments is secure so that both the epi and endo instruments may be stabilized when the magnetic alignment and ablation events occur.

Another specific example according to the present disclosure involves the above-characterized at least one epi magnet being among a first set or plurality of ring magnets, and the at least one endo magnet being among a second set or plurality of ring magnets. For at least one mode of ablation use, each set of ring magnets is paired and/or aligned in an array (e.g., Halback or Poly arrangement) to augment a magnetic field on one side of the array, such that the sets are positioned opposite one another and an opposing sides of the tissue structure.

Using this type of magnetic-pairing approach, the epi instrument may be located on a portion of a heart accessible via an open-chest region, the endo instrument may be introduced through the groin, and the endo instrument may be paired and aligned with the epi instrument. As paired, the cells near the first target site may then be ablated. In this manner, the epi and endo instruments may be secured for delivery of ablation energy without use of a vacuum to draw one or both the epi and endo instruments.

As with the above instruments, certain exemplary aspects of the present disclosure involve apparatuses (e.g., assemblies) including a pair of instruments used for tissue cell ablation such as but not necessarily limited to cardiac tissue. In one example, the instruments include an epi instrument and an endo instrument which (optionally) may be used with a control circuit for applying control over various forces involved. For example, these forces may include any one or a combination of: magnetic forces where electromagnetic energy may be used (e.g., with electrical energy delivered to at least one set of magnets); ablation-energy forces to control the energy delivery at the electrode in terms of magnitude; frequency and/or periodicity; and forces in the context of physical movement of one or both instruments such as for the purpose of positioning or repositioning the apparatus. The epi instrument has an ablation-delivery element and at least one epi magnet secured relative to the ablation-delivery element, and the endo instrument has at least one endo magnet and a mechanism (e.g., spherical or cylindrical shape) configured with the endo magnet to facilitate endo-instrument movement in a forward direction. The instruments may be moved towards a first target ablation site, and then to a second ablation site, while the at least one ablation-delivery element and the endo instrument are drawn towards one another via magnetic forces and on either sides of tissue structure. The ablation-delivery element and the endo instrument are cooperatively configured to move together via the magnetic forces for the ablation at the target site(s).

In a more specific example embodiment, the epi instrument has the one epi magnet as one of several epi magnets secured relative to the ablation-delivery element(s) that move with the magnet(s) of the endo instrument for locating at or near the second target site.

In certain embodiment involving use of a control circuit (e.g., with a programmed CPU and/or other logic circuitry), the magnetic forces which draw the instruments together may be controlled by the control circuit as a function of the tissue thickness, with increased forces applied for the thicker tissue. The magnitude of such forces may be determined empirically and/or be discerned based on feedback (e.g., monitoring effectiveness of the magnetic pairing or positioning before each ablation-application event).

The same control circuit or a different circuit may be used to apply physical forces to move one or both instruments (e.g., from a remote position) such as via a CPU-controlled surgical robot. Such physical forces may be applied, for example in such cases where at least one rail is used to secure one of the instruments, to the rail(s) with the magnetic forces being used to help maintain relative positioning of the instruments for a subsequent ablation event.

Consistent with the above examples, other examples of the present disclosure are directed to procedural aspects in which the epi and endo instruments may be used. In one such procedure, alignment of the magnetic array may be across myocardium for example, in connection with use of Design 2 and by a surgeon inserting an epi catheter (for presentation of the epi instrument) through a commercially available thoracic port. The surgeon or an electrophysiologist may then insert the endo catheter through a sheath and bring it in the vicinity of the epi-catheter magnetic array across the targeted tissue area. One of the above-discussed specific magnetic-array arrangement allows for self alignment of the magnets across the tissue. This alignment may be strongest only in one direction (e.g., especially with an augmented magnetic field such as provided by the Halback or Poly arrangement) which enables the self-alignment across the tissue as the magnetic arrays come close to each other across the tissue.

Further procedures also according to the present disclosure, may involve use of a rail and alternatively may not involve any such rail or guide. In one such instance, the epi catheter may be inserted via the ports on its own without aid of any guide (such as a rail) and positioned around the tissue anatomy of interest. Alternatively, the surgeon can also use a reference rail to define the path of the ablation on the surface of the heart and then feed the epi catheter over the reference rail. In both cases, the endo catheter will self-align with the magnetic array of the epi catheter as soon as it is brought close to the epi-catheter magnetic arrangement across the tissue.

In examples involving procedures using such a rail as a reference, the rail may provide a guiding path for the epi catheter. The epi catheter can also use a reference rail as a guiding path to achieve gap-free lesions along a path. Exemplary advantages of this approach include: the rail being used to provide a fixed reference point while the epicardial catheter is moving, thereby limiting likelihood of big movements or leaving gap(s) between adjacent ablation spots; ensuring continuous ablation along a single line; workflow optimization in that the surgeon introduces the guiding rail through thoracoscopic port, the surgeon positions the guiding rail around the pulmonary vein or the targeted anatomy, the epicardial catheter is then fed over the reference rail and the epi magnetic-ablation element is positioned at the start point. The electrophysiologist may then assist and/or permit for pairing of endo and epi magnetic elements for ablation, and the surgeon may start advancing the epi catheter along the reference rail and performing ablation.

In connection with the above example, the rail can be used to assist with automation of advancing the epicardial ablation elements along the ablation path. Such workflow may include the surgeon introducing the guiding rail through thoracoscopic port, and positioning the guiding rail around the pulmonary vein or the anatomy. The physician may feed an epicardial catheter over the reference rail and then position the epi magnetic-ablation element in the start point. The endo magnetic-ablation elements may then be paired. In an automated implementation involving a system including such epi and endo instruments, a control unit (e.g., computer-controlled step motor) is activated to slowly advance epi catheter along the reference rail until a point at or near cells to be ablated where the movement is stopped and ablation is performed. The ablation element(s) may then be advanced for another ablation and the process repeated.

In more specific embodiments of the present disclosure, the above aspects may be enhance by the above-characterized apparatuses including and/or procedures using, a sensor attached near or at the distal end of the epi instrument and/or endo instrument to provide feedback concerning thickness of the tissue area or areas which are targeted for the ablation. With such feedback, a controller may be used as part of the system (e.g., with the controller including logic circuitry such a programmable circuit or CPU) to cause controlled delivery of energy used in connection with an amount of magnetic pull across tissue areas as a function of the at least one thickness associated with tissue targeted for ablation.

Different approaches and/or sensors may be used (as alternatives or in combination) to provide such feedback/control. According to one aspect, a magnetic-field sensor is used to provide feedback in the form of magnetic-field strength measurement between the two magnetic elements while they are positioned across the ablation-targeted tissue. This measurement is used to define or characterize the thickness and from this characterized thickness, an amount of magnetic pull may be ascertained (e.g., via a lookup table in memory circuit of the controller) and the controller then applies this level of energy, so that the ablation procedure is effectively independent of the tissue thickness

According to another aspect, a contact force sensor is used instead of, or along with the above type of sensor, to characterize or indicate the tissue thickness. Different types of contact force sensors may be used in this regard, including concentric capacitive sensors and/or others (e.g., with the tissue being used as though a dielectric between electrodes of the capacitive sensor corresponding to conductive portions of the epi and endo instruments as oppositely positioned across the targeted tissue. Again, this measurement may be used to define or characterize the thickness and from this characterized thickness, an amount of controlled energy delivery may be ascertained such as described above.

As yet another aspect which is also accordance such specific aspects of the present disclosure, an impedance characterization is used instead of, or along with the above type of sensor(s), to characterize or indicate the tissue thickness. Such impedance characterization may be used to indicate the tissue thickness directly, or indirectly such as by indicating type of tissue. For such impedance characterization, using conductive portions of the epi and endo instruments as oppositely positioned across the targeted tissue, a small current is injected from one such electrode through the tissue and towards the other electrode. The tissue between the electrodes acts to resist or impede the flow of the current to a degree which is a function of the type of tissue and/or its thickness. This degree of impedance is sensed, via feedback as is the case with each of the above two types of sensors, by a wire or by wireless (e.g., radio frequency (RF) signaling such as passive RF identification circuitry, Bluetooth, etc.), and the impedance measurement may be used to define or characterize the thickness and from this characterized thickness, an amount of controlled energy delivery may be ascertained such as described above.

By using one or more such sensors and a communicatively-coupled controller in this regard, as a function of tissue thickness, a measurement of the tissue thickness may be captured so that consistent contact force is maintained independent of tissue thickness or variation.

An example, workflow in connection with such measurement(s) may be as follows: feedback from the contact force is forwarded to the controller which controls the magnetic strength (e.g., during a non-ablation period); the processor adjusts balloon size of the basket (e.g., Design type 2) or other mechanism that may introduce the appropriate gap size between the epi-endo magnetic array to maintain a consistent contact force independent of the tissue thickness (along the ablation path).

An example workflow via an alternate method may involve receiving feedback from the contact force as an input; communicating this feedback to a surgeon through a tactile feedback system, and once received the physician may then use the feedback and adjust magnetic strength accordingly such as by manual force and/or via step-wise control (e.g., on a button-type or graphic user interface which may be part of the controller).

As alternative or in addition to the above approaches, an automated modification may involving measuring or estimating the magnetic strength (e.g., interpolation based on feedback from other cardiac procedures and/or the targeted tissue at issue) along the ablation path and maintaining this measured/estimated magnetic strength as a consistent contact force which is applied independent of any further ascertained feedback of the tissue thickness; for example, with up to 1 mm, up to 5 mm and in some cases up to 8-10 mm of tissue-thickness variation being permitted as an exception. Uniform contact force can help achieve uniform ablation results along the ablation path independent of tissue thickness and operator training or skill level.

In alternative approaches consistent with the above in order to maintain a consistent contact force independent of the tissue thickness, also according to the present disclosure, the apparatus may include a pneumatic controller and the magnetic elements may be electromagnets, fluid magnets and/or above-characterized basket or another type of adjustable balloon structure to control the magnetic strength involving the epi and endo instruments. The control system is to adjust magnetic strength using these mechanisms as contact force information is collected from the sensors along the ablation path.

Below highlights the challenges with tissue variation: The first image highlights the level of trabeculation and uneven endocardial tissue thickness. Second image is a transmural image of the tissue to highlight the thickness variation across the ablation path. The third image is a combination of all three tissue types mentioned: regions where the tissue is relatively thin, regions with trabeculation, and then a region that is relatively thick. Our catheter smoothly moves over all three regions and is able to deliver consistent contact force and uninterrupted ablation to the tissue.

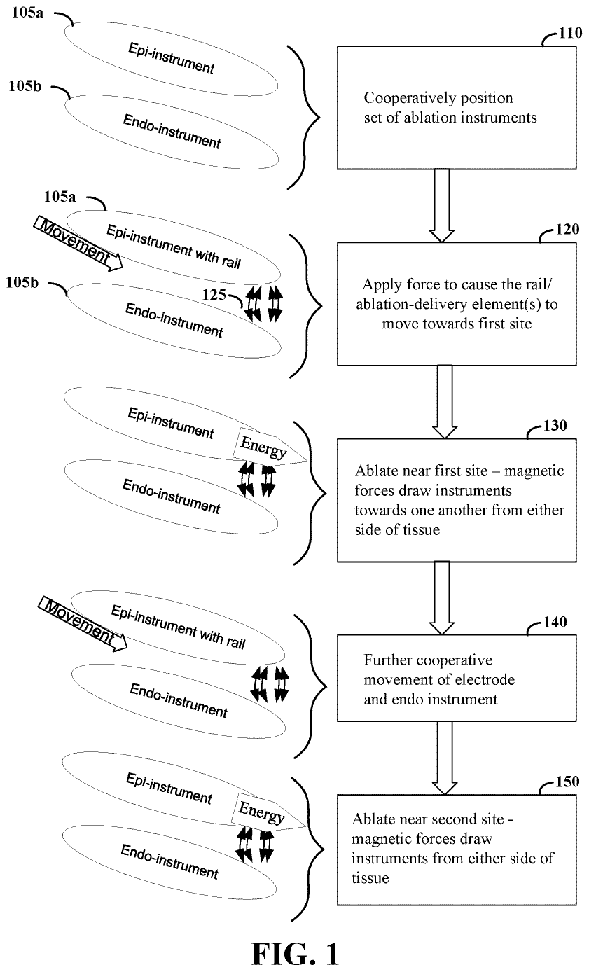

FIG. 1 is a flow diagram illustrating another more-specific manner in which such methodology, involving use of an epi instrument 105a and an endo instrument 105b as described above, may be carried out in practice as a procedure to test or treat a specific condition. In this one particular example method, the epi instrument 105a and the endo-instrument 105b are prepared, such as being cooperatively positioned as at block 110 (e.g., presented for introduction into the patient not yet magnetically coupled to one another) for an ablation procedure. In this specific illustration specific exemplary parts of each such instrument are not shown in FIG. 1; however, the epi instrument 105a may include an optional rail as discussed above, at least one ablation-delivery element and at least one epi magnet secured relative to the at least one ablation-delivery element, and the endo instrument 105b may include at least one endo magnet and a mechanism including a curved-or rounded-shape portion (e.g., an additional magnet with a rounded end or basket), and being configured with the at least one endo magnet to facilitate movement and/or positioning of the endo instrument 105b. In more specific embodiments, the basket may be used as a mechanism to facilitate endo-instrument movement. The basket may include a spherical-like expandable area containing the at least one endo magnet or at least one additional endo magnet having any of various shapes such as a spherical and/or cylindrical shape.

At block 120, FIG. 1 depicts a force being applied to cause the epi instrument and the at least one ablation-delivery element to move towards a first target site, where nearby cells are to be ablated while the (at least one) ablation-delivery element and the endo instrument 105b are drawn towards one another via magnetic forces 125 and on either sides of tissue structure. In different examples, this movement of the ablation-delivery element (or the epi instrument 105a) and the endo instrument 105b may involve movement of the instruments and/or relevant parts of the instruments being moved at least concurrently (or simultaneously), and/or the movement may be with such relevant parts of the instruments being moved in sequence such as with magnetic forces 125 aiding movement of the lagging instrument (or instrument part) in a pulling-along type manner.

At block 130, a first application of ablation energy may be applied for ablation of cells near the first target site occurs. At this juncture, the magnetic forces 125 draw the endo instrument 105b and the (at least one) ablation-delivery element of the epi instrument 105a towards one another while they are located on either sides of tissue structure.

Optionally, one or more further applications of ablation energy may be applied with or without repositioning the tissue-opposing positions of the epi instrument 105a and the endo-instrument 105b. For example, as shown in FIG. 1, in light of the cooperative design of the epi instrument 105a and an endo instrument 105b, the epi instrument 105a and the endo-instrument 105b may be moved from the first site to another, second site another ablation event. Accordingly, block 140 depicts cooperative movement of the at least one ablation-delivery element and the endo instrument (e.g., as discussed above) towards the second site while magnetic forces are engaged between the at least one epi magnet and the at least one endo magnet. If such forces momentarily disengage, assistance may be provided from another external force. As examples, this may be achieved by a force to push the instrument with a lagging position (e.g., its rail, cable, etc.) to advance the instrument, or pulling the lagging instrument (e.g., via a cable having a hook or other securing end-positioned component) from an end opposite the introduction entry point). After such repositioning at or near the second site, as depicted at block 150, the (at least one) ablation-delivery element may be used again to ablate cells in tissue structure while the epi instrument and the endo instrument are on opposing sides of the tissue structure near the second target site.

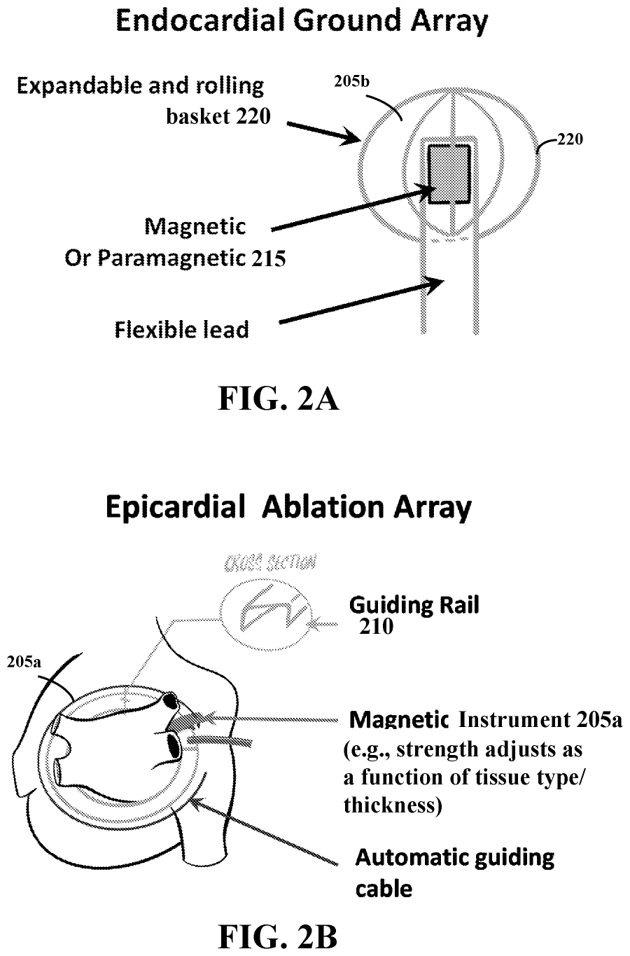

FIGS. 2A and 2B respectively show an endo instrument and an epi instrument of one type of exemplary ablation-directed apparatus. The epi instrument 205a (similar to 105a of FIG. 1) may include an introducer mechanism, such as a guiding/reference rail 210, at least one ablation-delivery element, at least one epi magnet secured relative to the at least one ablation-delivery element and mechanism to adjust magnetic strength (e.g. pneumatic or a balloon system), and the endo instrument 205b (similar to 105b of FIG. 1) may include at least one endo magnet (or paramagnet) 215 and a basket 220 (both elements 215 and more importantly 220 have a rounded shape for facilitating moving and positioning the endo instrument 205b. The instruments shown in FIGS. 2A and 2B may be used, for example, as a bipolar hybrid ablation system for aligned Afib (atrial fibrillation) transmural treatment.

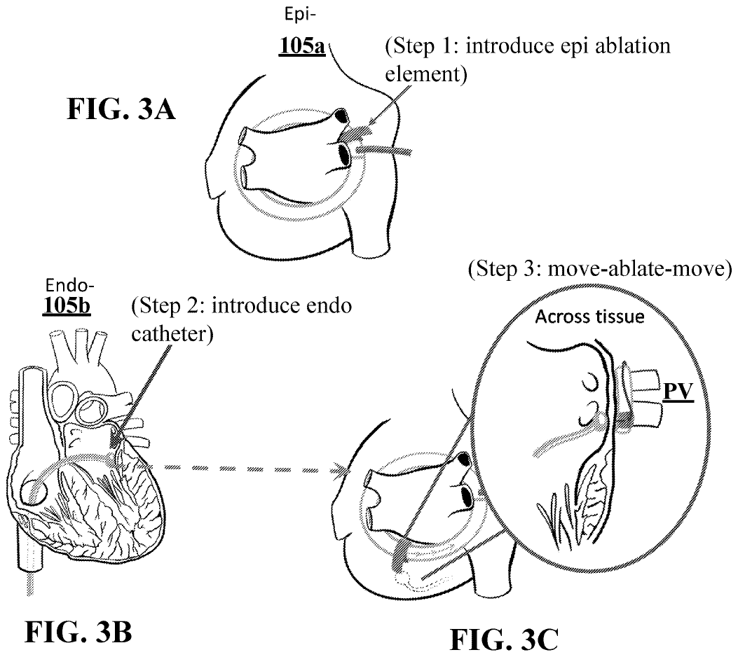

FIGS. 3A, 3B and 3C are related diagrams respectively showing steps of another example for use of an ablation-directed apparatus. As mentioned in connection with the above figures, FIG. 3A depicts a force being applied to advance introducer tube attached to rail and at least one ablation-delivery element to move around the ablation path, towards a first target site, where nearby cells are to be ablated. Next, the endo instrument 105b is introduced through the vasculature and brought close to the first target site across the tissue from epi instrument. The (at least one) epi ablation-delivery element and the endo instrument 105b are drawn towards one another via magnetic forces (e.g., 125 of FIG. 1) and on opposite sides of the tissue structure. In different examples, this movement of the ablation-delivery element (or the epi instrument 105a) and the endo instrument 105b may involve movement of the instruments and/or relevant parts of the instruments being moved at least concurrently (or simultaneously), and/or the movement may be with such relevant parts of the instruments being moved in sequence such as with the magnetic forces (e.g., 125 of FIG. 1) aiding movement of the lagging instrument (or instrument part) in a pulling-along type manner. A first application of ablation energy may be applied for ablation of cells near the first target site occurs. At this juncture, the magnetic forces 125 draw the endo instrument 105b and the (at least one) ablation-delivery element of the epi instrument 105a towards one another while they are located on either sides of tissue structure.

The epi instrument will be guided and repositioned at the second site of ablation. The magnetic field, impedance or contact force will be measured to characterize tissue type and thickness prior to ablation. If magnetic contact force with tissue is not consistent with the prior position, then magnetic force adjustment mechanism will be used to modify strength prior to ablation or ablation energy parameters will be altered.

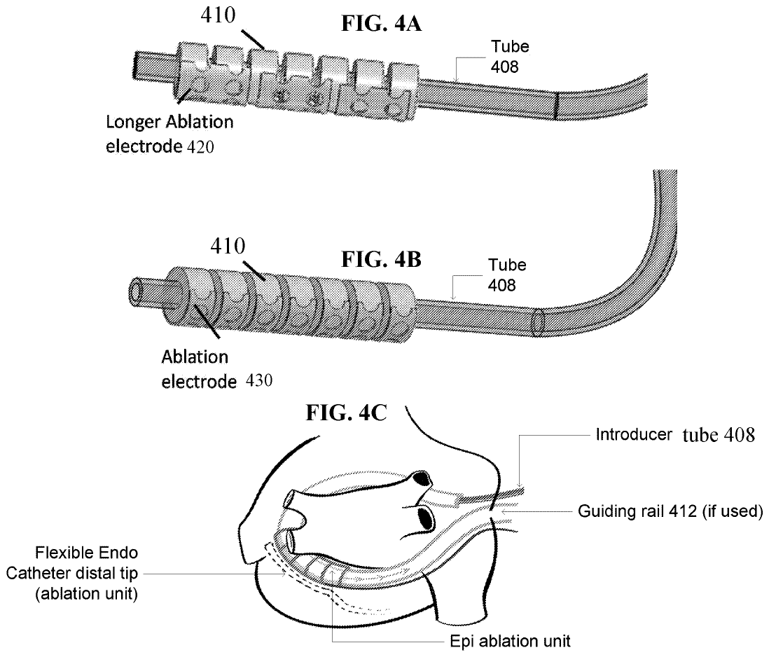

Cooperative movement of the at least one ablation-delivery element and the endo instrument (e.g., as discussed above) towards the next ablation site will continue while magnetic forces are engaged between the at least one epi magnet and the at least one endo magnet, until ablation path of interest is complete. FIGS. 4A, 4B and 4C are perspective views of exemplary parts of epi instruments which may be used in connection with a type of exemplary ablation-directed apparatus in which the epi instrument (105a of FIG. 1) includes an introducer mechanism such as tube 408, an optional guiding/reference rail 412, at least one ablation-delivery electrode or element, One or more epi magnets 410 may be configured as a set of magnets and arranged in a sequence. The magnets 410 may be ring shaped or of another shape and/or type, and may be as secured relative to at least one ablation-delivery elements and mechanism are provided to adjust magnetic strength (e.g. a balloon). Further, the endo instrument may include at least one endo magnet or a set of magnets in a sequence and may be configured with the at least one endo magnet and/or arranged in an opposing magnetic sequence to facilitate positioning of the endo instrument (105b of FIG. 1).

FIGS. 4A and 4B are different in that FIG. 4A shows a longer ablation electrode 420 over multiple electrodes, while FIG. 4B shows another example in which there is a one-to-one correspondence between the magnets 410 and the electrodes 430.

Similar to FIG. 3, in FIGS. 4A and 4B, a force is applied to advance introducer tube attached to maybe a reference rail and at least one ablation-delivery element to move around the ablation path, towards a first target site, where nearby cells are to be ablated. Next, the endo instrument 105b is introduced through the vasculature and brought close to the first target site across the tissue from epi instrument. The (at least one) epi ablation-delivery element and the endo instrument 105b are drawn towards one another via magnetic forces 125 and on either sides of tissue structure. In different examples, this movement of the ablation-delivery element (or the epi instrument 105a) and the endo instrument 105b may involve movement of the instruments and/or relevant parts of the instruments being moved at least concurrently (or simultaneously), and/or the movement may be with such relevant parts of the instruments being moved in sequence such as with magnetic forces 125 aiding movement of the lagging instrument (or instrument part) in a pulling-along type manner.

A first application of ablation energy may be applied for ablation of cells near the first target site occurs. At this juncture, the magnetic forces 125 draw the endo instrument 105b and the (at least one) ablation-delivery element of the epi instrument 105a towards one another while they are located on either sides of tissue structure.

The epi instrument will be guided and repositioned at the second site of ablation. The magnetic field, impedance or contact force will be measured to characterize tissue type and thickness prior to ablation. If magnetic contact force with tissue is not consistent with the prior ablation position, then magnetic force adjustment mechanism will be used to modify strength prior to ablation or ablation energy parameters will be altered. Cooperative movement of the at least one ablation-delivery element and the endo instrument (e.g., as discussed above) towards the next ablation site will continue while magnetic forces are engaged between the at least one epi magnet and the at least one endo magnet, until ablation path of interest is complete.

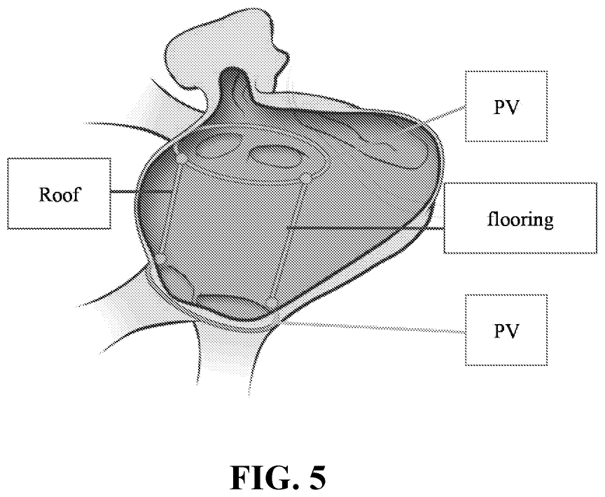

FIG. 5 is a heart-related diagram illustrating application, from an endocardium perspective, of using the second type of apparatus showing pathways for treatment in connection with left atrium (LA) posterior box lesion, according to certain exemplary aspects of the present disclosure. As pulmonary veins (PVs) are identified as the most common site for triggering of atrial fibrillation, Pulmonary Vein Isolation (PVI) is the main treatment of atrial fibrillation or AF. This type of AF treatment involves boxing and isolating the pulmonary veins which results in LA posterior box lesion similar to the ablation path in FIG. 5. A major cause of AF Recurrence is failure to create full thickness transmural lesions across the tissue or inducing continuous lesions (gap-free lesions).

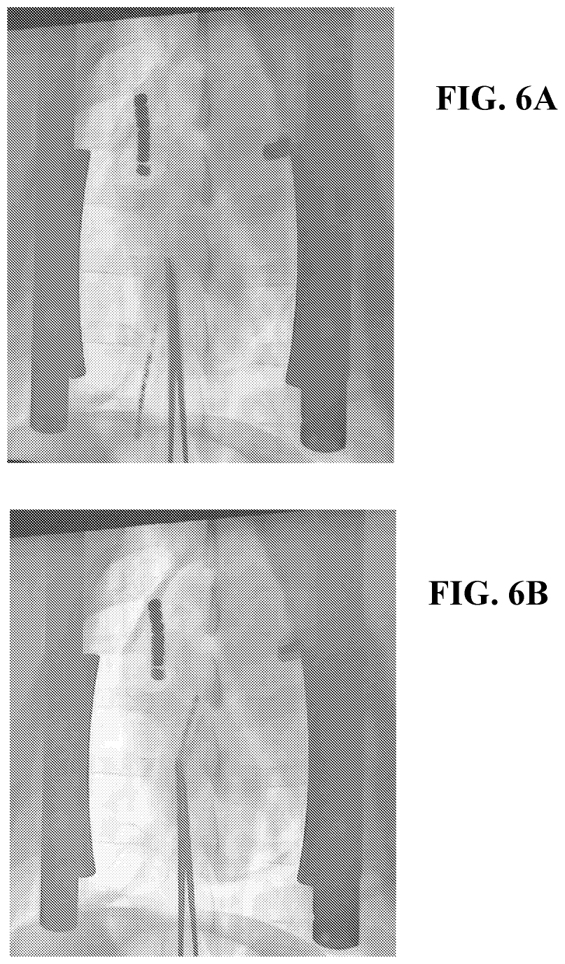

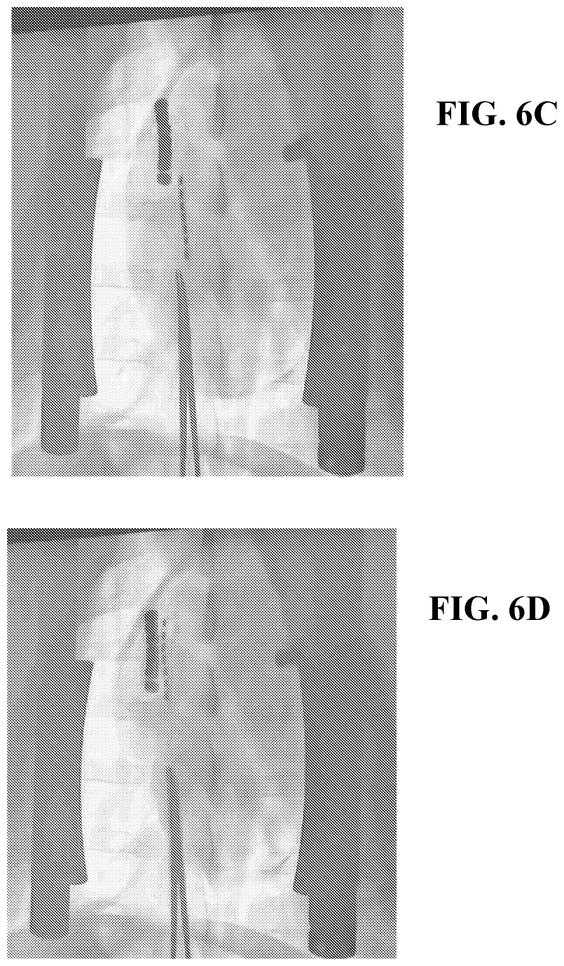

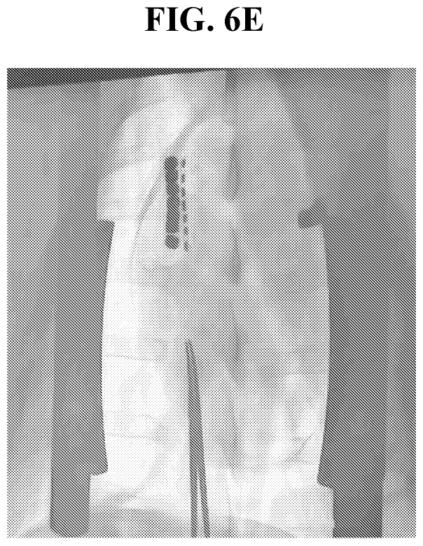

FIGS. 6A, 6B, 6C, 6D, and 6E are related diagrams (in X-ray form) depicting catheter application of the second type of apparatus with each of these diagrams showing sequential progress of the apparatus'epi instrument positioned in the first target site, where nearby cells are to be ablated and endo instrument being advanced through a pig model to a complementarily-constructed (or epi) instrument for an epi-endo-paired positioning on either side of targeted tissue before delivery of ablation energy, according to certain exemplary aspects of the present disclosure.

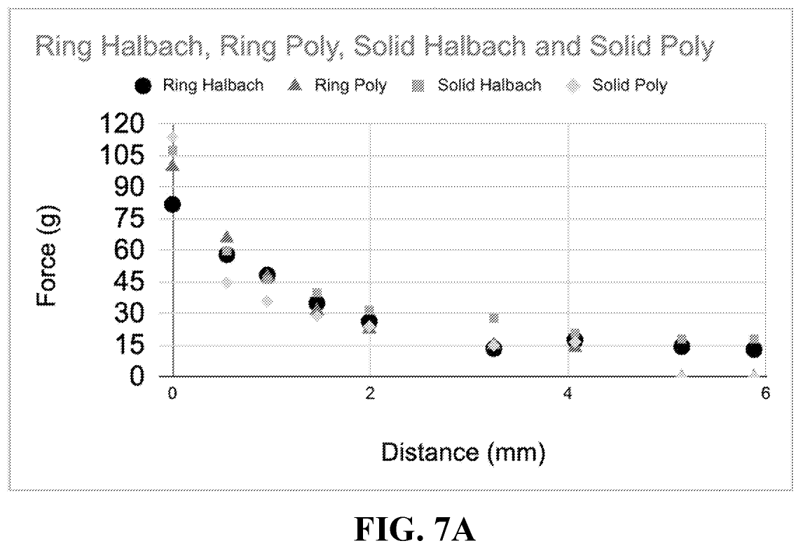

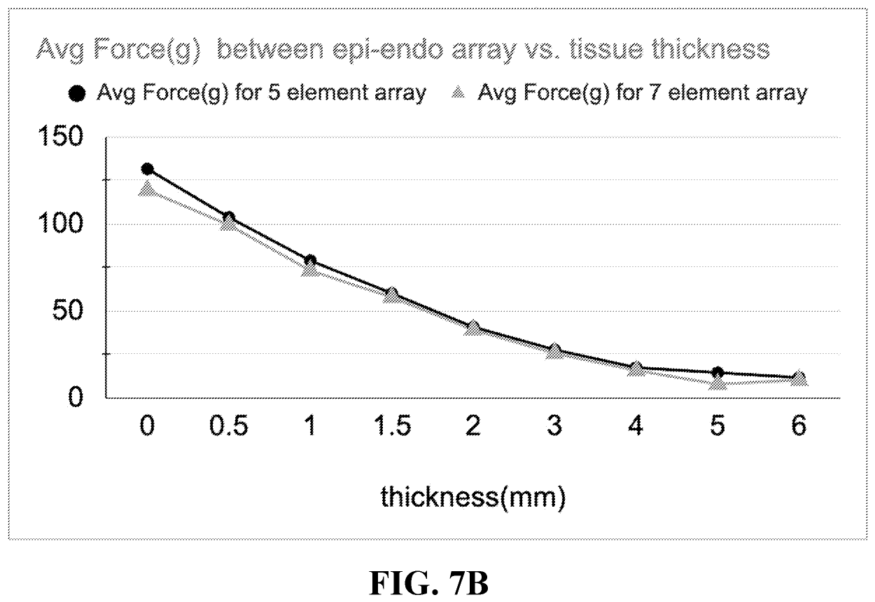

FIGS. 7A and 7B are respective graphs showing different contact force studies validating performance criteria in an experiment using another, second type of apparatus including epi and endo instruments consistent with the above discussion. The exemplary ablation-directed apparatus of FIG. 7A shows various forces versus distance (tissue thickness) associated with different magnetic-type structures, varying magnet design and magnet set arrangement. FIG. 7B showing an example of a five-element ring-array design in comparison to a seven-element ring-array design, and showing effects as the distance/tissue thickness increases.

In certain detailed experimental examples involving epi and endo instruments configured as discussed and illustrated above, successful treatment outcome may be realized via a single and cost effective procedure. This in contrast to treatment outcomes of patients with persistent atrial fibrillation (AF) for which multiple treatments are often applied when using previous approaches and technology. In experimental procedures and according to the above examples of the present disclosure, significant improvement in this AF patient group compared to limited effectiveness of endo-catheter ablation involving previously-known approaches.

Moreover, such procedures according to certain aspects of the present disclosure provide significant improvement in terms of safety. This follows as fewer and less severe complications and adverse effects or adjacent tissue damage results during ablation. Because the ablation elements are targeted and focused on the tissue between the two magnetic arrays, the chances of adverse effect to adjacent tissue damage (such as esophageal) is minimized or eliminated.

The following is an example characterization of such experimental (two-minimally invasive) procedures according to the present disclosure. The minimally-invasive procedures are catheter ablation and thoracoscopic guided ablation using special surgical tools. The thoracoscopic and catheter ablations are performed at different points in time to ablate from endocardial and epicardial surfaces, respectively, in two separate procedures which are time consuming and do not guarantee alignment of the endocardial and epicardial lesions, limiting the ability to create full-thickness lesions. Thus, a significant opportunity is to ablate both sides of the heart wall in one procedure to guarantee transmurality in patients with longstanding AF and result in better treatment.

By using the above-described types of paired epi and endo instruments, according to the present disclosure, patient populations who need multiple treatments using current technology may be treated by ablation for successful long-lasting treatment in a single procedure. In this manner, the need for multiple patient recovery periods (e.g., costing a payer up to and sometime over $40,000 associated with medication cost, second procedure costs, and hospitalization per patient due to reoccurrence) may be eliminated. More specifically, the above-described types of paired epi and endo instruments, according to the present disclosure, are designed to overcome various technical problems such as: incomplete ablation paths and the resultant adverse outcomes of these incomplete ablation paths; rigidity of endocardial catheters; and inconsistent contact force along the ablation path.

Accordingly, many different types of treatments and devices using such instruments may be advantaged by applications and aspects of the present disclosure, and these include the above-discussed aspects and exemplary treatments involving treatments of cardia arrhythmias and others (including but not limited to the related examples in the above-identified U.S. Provisional Application (STFD.423P1). In connection with other types of treatments, applications and aspects of the present disclosure are directed to types of treatment including, for example, treatment of chronic pain in the neck, back, knee, pelvic and peripheral nerve pain, as may be achieved by preventing transmission of pain signals. In yet other examples, applications and aspects of the present disclosure may be used for treatment of certain types of foreign objects obstructing internal passages such as urinary-related obstructions having certain chemistries which may be reduced by application ablation energy, and/or for transperineal laser ablation treatment in the lower urinary tract and other tissue areas. As may be apparent in light of certain surgical-application areas (e.g., areas other than the specific cardia examples as illustrated in the present disclosure), the terms “epi” and “endo” are not necessarily indicative of where the instruments are to be placed. For example, the epi-instrument (having one or more ablation electrode elements) may be used at an inside position relative to the endo-instrument, with the endo-instrument being used at an outside position relative to the epi-instrument. Using such an epi-instrument in this manner may be applicable, for example, to a urinary tract application in which the ablation electrode element(s) are to be used inside the urinary tract while the endo-instrument is located outside the urinary tract.

It is recognized and appreciated that as specific examples, the above-characterized figures and discussion are provided to help illustrate certain aspects (and advantages in some instances) which may be used in the manufacture of such structures and devices. These structures and devices include the exemplary structures and devices described in connection with each of the figures as well as other devices, as each such described embodiment has one or more related aspects which may be modified and/or combined with the other such devices and examples as described hereinabove may also be found in the Appendix of the above-referenced Provisional.

For example, in certain of the above-discussed embodiments, one or more modules are discrete logic circuits or programmable logic circuits configured and arranged for implementing these operations/activities, as may be carried out in the approaches described in the Appendix, showing how one and/or both parts may be directed, under control of a CPU, towards the target tissue (e.g., via a pathway such as a femoral artery towards the heart). In such an example the CPU may operate as a series of steps according to programmed instructions which might be based on feedback (e.g., such as sensors inside or outside the patient's heart and/or based on feedback via visualization on display monitors which show imaging inside the patient's heart during insertion procedures.

Certain specific examples, relating to the above-described aspects, are directed to a computer program product (e.g., non-volatile memory device), which includes a machine or computer-readable medium having stored thereon such programmed instructions to be executed by a computer (or other electronic device/logic circuit) to perform such steps (or operations/activities). In such CPU-related embodiments, a programmable circuit may be implemented as one or more computer circuits, including memory circuitry for storing and accessing a program to be executed as a set (or sets) of instructions (and/or to be used as configuration data to define how the programmable circuit is to perform), and an algorithm or process as described above is used by the programmable circuit to perform the related steps, functions, operations, activities, etc. Depending on the application, the instructions (and/or configuration data) can be configured for implementation in logic circuitry, with the instructions (whether characterized in the form of object code, firmware or software) stored in and accessible from a memory (circuit).

Based upon the above discussion and illustrations, those skilled in the art will readily recognize that various modifications and changes may be made to the various embodiments without strictly following the exemplary embodiments and applications illustrated and described herein. For example, methods as exemplified in the Figures may involve steps carried out in various orders, with one or more aspects of the embodiments herein retained, or may involve fewer or more steps. Such modifications do not depart from the true spirit and scope of various aspects of the disclosure, including aspects set forth in the claims.

What is claimed:

1. An apparatus comprising:
a set of ablation instruments including
an epi instrument having at least one ablation-delivery element and at least one epi magnet secured relative to the at least one ablation-delivery element, and
an endo instrument having at least one endo magnet and being configured with the at least one endo magnet to facilitate positioning of the endo instrument;
the epi instrument and the endo instrument being configured to respond to an applied force that causes the at least one ablation-delivery element to move towards a first target site where nearby cells are to be ablated while the at least one ablation-delivery element and the endo instrument are drawn towards one another via magnetic forces and on either sides of tissue structure; and
the at least one ablation-delivery element and the endo instrument being configured to cooperatively move towards a second target site while magnetic forces are engaged between the at least one epi magnet and the at least one endo magnet, and to ablate cells in tissue structure near the second target site where nearby cells are to be ablated while the at least one ablation-delivery element and the endo instrument are drawn towards one another via magnetic forces and on either sides of tissue structure near the second target site.

2. The apparatus of claim 1, wherein the at least one epi magnet secured relative to the at least one ablation-delivery element move with the at least one endo magnet, in response to the movement towards the second target site, and the apparatus further including:
a sensor to provide feedback concerning at least one thickness of the tissue area or areas targeted for ablation, and
a controller, including logic circuitry to cause controlled delivery of energy used in connection with magnetic pull across tissue areas as a function of said at least one thickness.

3. The apparatus of claim 1, further including a control circuit, including logic circuitry, to control the magnetic forces, and including an elongated mechanism for movement within anatomy having the tissue, wherein the mechanism refers to or includes: a lumen, a catheter, and/or a spherical-like basket containing the at least one endo magnet or at least one additional endo magnet having a spherical or cylindrical shape.

4. The apparatus of claim 1, further including a control circuit, including logic circuitry, to provide control over the magnetic forces as a function of thickness of the tissue structure.

5. The apparatus of claim 1, wherein the at least one epi magnet and the at least one ablation-delivery element, while being secured relative to one another, are to be fixedly secured by a rail while used in a tissue-presentation mode which precedes a mode in which the at least one ablation-delivery element is to apply energy for ablating cells.

6. The apparatus of claim 1, wherein the at least one epi magnet and the at least one ablation-delivery element, while secured relative to one another, are to move along a pathway to facilitate the movement of the at least one ablation-delivery element towards a second target ablation while magnetic forces remain engaged between the at least one epi magnet and the at least one endo magnet.

7. The apparatus of claim 1, further including a vacuum to draw the at least one ablation-delivery element against the tissue structure.

8. The apparatus of claim 1, wherein further including an expandable basket in which the at least one endo magnet resides, wherein the expandable basket is to expand and is to provide a conduction electrode while the at least one ablation-delivery element is to ablate the cells.

9. The apparatus of claim 1, further including an expandable basket in which the at least one endo magnet resides, wherein the expandable basket is free to rotate while the endo instrument is moved along tissue surface.

10. The apparatus of claim 1, further including an expandable, rotatable basket in which the at least one endo magnet resides, wherein the expandable, rotatable basket is to permit the expandable basket is to rotate and facilitate the at least one endo magnet to be pulled along by the at least one epi magnet in response to the magnetic forces, and is further to provide a ground reference while the at least one ablation-delivery element applies energy for ablating the cells.

11. The apparatus of claim 1, wherein the at least one epi magnet includes a plurality of magnets associated with the epi instrument, and the at least one endo magnet includes a plurality of magnets associated with the endo instrument, wherein for at least one mode of ablation use, the plurality of magnets associated with the epi instrument is to be paired and/or aligned in an arrangement opposite the plurality of magnets associated with the endo instrument on an opposing side of the tissue structure.

12. The apparatus of claim 11, further including an additional endo magnet having a curved outer surface, wherein each of the at least one endo magnet and the additional endo magnet form a series of magnets in an array configured and/or used to augment a magnetic field on one side of the array, and wherein the at least one ablation-delivery element includes an ablation-energy-delivery component, or electrode, secured to respective surfaces of a plurality of immediately adjacent ones of the set of magnets arranged in a sequence.

13. The apparatus of claim 1, further including an additional endo magnet having a curved outer surface, wherein the apparatus further comprises:
a set of sequentially-arranged endo magnets including the at least one endo magnet and the additional endo magnet, and
a set of sequentially-arranged epi magnets including the at least one epi magnet and the additional epi magnet, and wherein each magnet of one or both sets of magnets is a ring magnet situated at least partially around an elongated support.

14. The apparatus of claim 13, wherein the elongated support refers to or includes a guide wire, carrier or tube, and wherein each magnet of at least one of the sets of sequentially-arranged magnets, refers to or includes a ring magnet.

15. The apparatus of claim 1, further including a sensor to provide feedback concerning at least one thickness of the tissue area or areas targeted for the ablation, and a controller, including logic circuitry to cause controlled delivery of energy used in connection with magnetic pull across tissue areas as a function of the at least one thickness associated with tissue targeted for ablation.

16. The apparatus of claim 1, wherein the epi and endo instruments are configured to be secured for delivery of ablation energy without use of a vacuum to draw either of the epi or endo instruments against the tissue structure.

17. A method comprising:
cooperatively positioning a set of ablation instruments including
an epi instrument at least one ablation-delivery element and at least one epi magnet secured relative to the at least one ablation-delivery element, and
an endo instrument having at least one endo magnet and being configured with the at least one endo magnet to facilitate positioning of the endo instrument;
applying a force to cause the at least one ablation-delivery element to move towards a first target site, where nearby cells are to be ablated while the at least one ablation-delivery element and the endo instrument are drawn towards one another via magnetic forces and on either sides of tissue structure;
ablating, via the at least one ablation-delivery element and the endo instrument, cells near the first target site while the magnetic forces draw the at least one ablation-delivery element and the endo instrument towards one another while being located on either sides of tissue structure;
causing cooperative movement of the at least one ablation-delivery element and the endo instrument towards a second target site while magnetic forces are engaged between the at least one epi magnet and the at least one endo magnet; and
using the at least one ablation-delivery element to ablate cells in tissue structure near the second target site while the epi instrument and the endo instrument are on opposing sides of the tissue structure near the second target site.

18. The method of claim 17, wherein the endo instrument further has an expandable, rotatable basket in which the at least one endo magnet resides, the expandable, rotatable basket rotates and facilitates the at least one endo magnet to be pulled along by the at least one epi magnet in response to the magnetic forces, and further, while the at least one ablation-delivery element applies energy to ablate the cells, the expandable, rotatable basket provides a ground reference.

19. The method of claim 18, further including introducing the epi instrument thoracoscopically while the at least one ablation-delivery element and/or the at least one epi magnet are secured to one another via a rail, cable, lumen or catheter.

20. The method of claim 17, wherein the at least one epi magnet includes a plurality of ring magnets, and the at least one endo magnet includes a plurality of ring magnets, wherein for at least one mode of ablation use, the plurality of ring magnets of the epi instrument is paired and/or aligned in an arrangement to augment a magnetic field on one side of the array, opposite the plurality of ring magnets of the endo instrument on an opposing side of the tissue structure.

21. The method of claim 20, wherein the at least one ablation-delivery element includes an ablation-energy-delivery component, or electrode, secured to respective surfaces of a plurality of immediately adjacent ones of the set of magnets arranged in a sequence.

22. The method of claim 20, further including: locating the epi instrument on a portion of a heart accessible via an open-chest region; introducing the endo instrument through the groin and causing the endo instrument to pair and align with the epi instrument; and then ablating the cells near the first target site; wherein the epi and endo instruments are configured to be secured for delivery of ablation energy without use of a vacuum to draw or secure a part of either the epi instrument or the endo instrument against tissue near the first target site for positioning to ablate the cells.
